(19) United States
(12) Patent Application Publication
(10) Pub. No.: US 2023/0207107 A1
(43) Pub. Date: Jun. 29, 2023

(54) Medical Treatment System for the Identification and Authorization of Healthcare Professionals

(71) Applicant: Fresenius Medical Care Deutschland GmbH, Bad Homburg (DE)

(72) Inventors: Carsten Mueller, Euerbach (DE); Michael Thorwarth, Frankfurt am Main (DE); Timo Speth, Schwebheim (DE); Karin Helga Trauner, Schweinfurt (DE); Ralf Mueller, Bad Homburg (DE); Qingsong Miao, Frankfurt am Main (DE)

(21) Appl. No.: 17/923,440

(22) PCT Filed: May 10, 2021
(86) PCT No.: PCT/EP2021/062272

§ 371 Date: Nov. 4, 2022

(30) Foreign Application Priority Data
May 11, 2020 (EP) 20173956.2

(51) Int. Cl. G16H 40/20; G16H 40/63; G06F 21/34

(52) U.S. Cl. CPC G16H 40/20; G06F 21/34; G16H 40/63

(57) ABSTRACT

The present disclosure relates to a medical treatment system with a reading device programmed for - preferably contactless - gathering of information which serves to identify a healthcare professional and programmed to provide the gathered information to an authorization device. Furthermore, the medical treatment system comprises an apparatus of a group of items consisting of one or more treatment apparatuses for treating a patient, one or more diagnostic apparatuses for examining the patient and one or more further clinic apparatuses, e.g., for the logistical running of the clinic or practice operation. Furthermore, the medical treatment system comprises an authorization device for allowing or prohibiting one or more activities with or on the apparatus from the group and/or one or more movements of the healthcare professional in the vicinity of the apparatus from the group depending on the information provided by the reading device.

### CROSS-REFERENCE TO RELATED APPLICATIONS

The present application is the national stage entry of International Patent Application No. PCT/EP2021/062272, filed on May 10, 2021, and claims priority to Application No. EP 20173956.2, filed in the European Patent Office on May 11, 2020, the disclosures of which are expressly incorporated herein in their entirety by reference thereto.

### TECHNICAL FIELD

The present disclosure relates to a medical treatment system.

### BACKGROUND

The modern health care system enables unprecedented medical care for today’s patients, not least through the use of complex diagnostic and treatment apparatuses. Their proper operation requires not only specific medical knowledge but also technical knowledge. Patient safety can usually only be guaranteed in connection with such complex diagnostic and treatment apparatuses if it is ensured that unauthorized persons are excluded from the intentional or unintentional operation of such apparatuses, or if it is ensured that this group of people does not actually have access to such or other medical apparatuses.

As an example of a complex treatment apparatus from practice, a device for the extracorporeal blood treatment by dialysis should be mentioned. In this type of blood treatment, blood is taken from the patient and guided extracorporeally along a blood circuit through a blood filter of the dialysis apparatus. During the blood treatment session carried out in this way, a large number of treatment parameters that can be set on the dialysis machine can be used to influence the treatment, inter alia by changing flow rates and/or drug dosages. It is not necessary to explain that such adjustments should advantageously only be made by a select, sufficiently qualified group of people.

Moreover, the access of unauthorized persons to patient rooms, treatment rooms, isolation wards, medical apparatuses and the like can already be strictly limited, to protect both the unauthorized person (keyword: isolation ward) and the patient (keyword: transplant ward), but also for reasons of patient privacy and/or data protection.

It is therefore desirable to protect medical treatment systems, i.e., above all devices for treating or examining patients, and the installation locations of such apparatuses, such as hospitals or practice rooms, against access or entry by unauthorized persons.

### SUMMARY

An aspect of to the present disclosure is achieved by a medical treatment system (or “medical set”) as disclosed herein.

The present disclosure relates to a medical treatment system comprising a reading device, a medical apparatus, and an authorization device.

The reading device is preferably designed as a read-in and/or read-out device. It is programmed to gather or capture data or information (hereinafter also referred to as information), preferably without contact. This capturing can be a reading-in or reading-out of the information.

The information gathered or captured by the reading device serves to identify clinic personnel, also referred to herein as healthcare professionals, such as a doctor, a nurse, a technicians, or other people from the clinic or practice environment, which may, in particular, herein also include patients or visitors.

Furthermore, the reading device is programmed to provide the gathered information, for example, to transmit the information to the authorization device as further described below or to have the information read out by the authorization device or to provide or transmit the information to the authorization device in another way.

Identification particularly means herein, to recognize a person or their authority with sufficient accuracy, i.e., as clearly as possible, i.e., to establish his/her identity and/or authenticity, as this is in practice the aim of, for example, a passport control. “Identify” is used here synonymously to “authenticate” and “recognize”. These terms are thus interchangeable. “Authorize” is synonymous with the determination of an authorization after an identification. In this context, identification/authentication always precedes subsequent authorization. Differences in meaning can be clearly seen in the respective context.

Identifying a person may be understood in several embodiments to mean that a person may be assigned, e.g., based on the identification device, to a group of people, e.g., healthcare professionals, medical staff, nursing staff, trainees, interns, administrative staff, facility management persons, or the like. A group of healthcare professionals, e.g., as set forth above, may be assigned certain powers as a whole, for example, entering predetermined areas, operating predetermined treatment or diagnostic devices, or the like, which may be stored in the authorization device, the reading device, and/or in the storage device of the medical treatment system. For example, an intern, a room attendant or a facility manager should not be able to operate an X-ray machine, trainees could hereby be allowed access to certain areas only when accompanied by a trained nurse, etc. It is therefore not always necessary to identify a specific person (e.g., Mr. Muller) via the reading device. In some embodiments described in the present disclosure, it may be provided for and sufficient to identify to which of the, e.g., above-mentioned, groups the specific person belongs.

The identification may be carried out via the reading device.

Furthermore, the medical apparatus is an item of a group of items consisting of one or more treatment apparatuses for treating a patient, one or more diagnostic apparatuses for examining the patient and one or more further clinic apparatuses, e.g., for the logistical running of the clinic or practice operation.

The previously referred to authorization device is programmed or configured to allow or prohibit one or more activities with or on the medical apparatus from the above-mentioned group and/or one or more movements of the healthcare professional in the vicinity or area of the medical apparatus of the above-mentioned group, in particular by activating or blocking hospital devices, for example doors. The allowing or prohibiting is carried out depending on the result of an evaluation of the information provided by the reading device. The evaluation may already be carried out by the reading device, alternatively, for example, by the authorization device.

Allowing may be enabling, authorizing or permitting an activity or movement, as well as unlocking and/or unsealing for example, a device such as a door, lock, computer access, etc. As used herein, authorize means to fully allow someone to perform what is here an activity or movement, to enable them to carry out or to give them permission for something, here the activity or movement.

Prohibiting can be an non-enabling a denial, a non-authorization or a non-permission, as well as a blocking, an shutting out and/or a locking.

Movements may include movements or advances by the healthcare professional in one direction, entering into an area by the healthcare professional, entering a room and/or otherwise moving, particularly in relation to the medical apparatus or its position. Allowing or prohibiting movements can be understood as granting or denying freedom of movement, at least to a predetermined place or space and/or into a predetermined place or room.

Activities may include the operation or use of the medical apparatus.

The authorization device can thus authorize activities on the medical apparatus, such as starting, setting or modifying a predetermined workflow to be performed or performed via the medical apparatus.

According to the present disclosure, the authorization device can use suitable actuators, of which only a few examples are given here, such as password-protected screen savers, software programs or touch screen surfaces, locks, door openers, etc. to allow or prohibit activities or movements,

Whenever “programmed” or “configured” is mentioned herein, it is also disclosed that these terms are interchangeable with one another.

Whenever “programmed” or “configured” is mentioned herein, it is also disclosed that the relevant apparatus is designed, in some embodiments, to automatically perform steps for which it is programmed or configured.

If a step or an action is disclosed herein, it is also disclosed that at least one apparatus of the medical treatment system is programmed or configured in several embodiments to perform the step or action.

Embodiments according to the present disclosure may comprise several, some or all of the following features in any combination, unless the person skilled in the art recognizes a specific combination as technically impossible.

In all of the preceding or following statements, the use of the expression “may be” or “may have” etc. is synonymous to “is preferably” or “has preferably,” etc. respectively, and is intended to illustrate embodiments according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” as encompassing “at least one”. This understanding is also equally encompassed by the present disclosure as the interpretation that a numeric word, for example, “one” may alternatively mean “exactly one”, wherever this is evidently technically possible for the person skilled in the art. Both understandings are encompassed by the present disclosure and apply herein to all used numerical words.

Whenever an embodiment is mentioned herein, it is then an exemplary embodiment according to the present disclosure.

In some embodiments the reading device is or comprises a sensor arrangement, in particular a card reader, a radar emitter and/or radar receiver, a camera and so forth or any combination of these and all further herein referred to sensors, scanners, receivers, readers, and other devices or apparatuses.

In several embodiments, the reading device is or comprises a finger print scanner for scanning finger prints, a device to recognize a movement pattern, gestures, biometric data or signatures or iris structure. Biometric data or signatures include, for example, fingerprints, facial features, iris structures, movement patterns and/or language signatures.

For this purpose, the reading device is preferably designed as a camera, as a emitter and/or receiver of radar waves, as a card reader for magnetic or chip cards, as a microphone for recording and/or analysing the voice of the healthcare professional (preferably with corresponding speech recognition), as an input device configured for inputting personal knowledge (via display or monitor, via haptic input devices, for example at least one touchpad or at least one keyboard), or comprises the like.

Data or information stored on the identification device which the healthcare professional optionally carries with them shall preferably be gathered via a camera or other suitable and programmed recognition devices or receiving devices of the reading device and/or transmitted by WiFi, Bluetooth or RFID, in particular as disclosed herein.

Information gathered using the above-mentioned designs of the reading device in the use of the medical treatment system can be understood here as data and information (in short: information) which serve to identify a healthcare professional.

Such data and information may be and/or will be stored on at least one server, database and/or data storage (device) and/or is or will be compared with data on at least one server, (a) database and/or data storage (device). A database may be located on a dedicated database server, on a personal computer or on another device. The data may be stored and/or will be stored unencrypted or encrypted.

In several embodiments the reading device is in cable and/or wireless connection to at least a server, a database and/or a data storage (device).

In some embodiments, the authorization device is in wired and/or wireless connection to at least a server, a database and/or a data storage (device).

In several embodiments, the connecting of the reading device and/or the authorization device and/or another device and/or apparatus mentioned herein to at least one server, database and/or data storage (device) takes place via at least one network, either wired or wireless.

Radar, in so far as it is mentioned here, is the abbreviation for Radio Detection and Ranging (for example: radio-based positioning and distance measurement). This term covers various detection and location procedures for objects (including people) based on electromagnetic waves in the radio frequency range (radio waves). As far as it is not obviously technically impossible for a person skilled in the art, the radar mentioned here also includes lidar (light detection and ranging) based on laser beams.

The radar emitter of the radar unit emits a bundled electromagnetic wave as a so-called primary signal. This is reflected by objects and the echoes are received by the radar receiver of the radar unit as a so-called secondary signal. The received echoes can be evaluated according to predetermined criteria. Among other things, the following information can thereby be obtained:
angle or direction to the (reflecting) object (or: subject);
distance to the object (using the time difference between emission and reception);
relative movement between emitter and object (using the Doppler effect from the frequency shift);
absolute speed of the object (by stringing together several individual measurements);
contours or images of the object.

In some embodiments, the reading device is programmed to capture a gait pattern, gait sample, movement profile, gestures or similar of the healthcare professional as information.

In some embodiments, the medical treatment system further has an identification device. The identification device is programmed and/or configured to be worn or carried by the healthcare professional on their body.

Thus, if the medical treatment system also has an identification device, the reading device may be programmed in these embodiments in a way that it can gather and/or transmit information stored on the identification device. Alternatively, relevant information can already be gathered by determining that the healthcare professional is carrying a specific, predetermined identification device or an identification device of a predetermined form or type, regardless of whether or not this identification device provides information concerning the identity of the specific healthcare professional carrying it.

In some embodiments, the reading device is configured to read an identification device carried by the healthcare professional, for example using radar technology, or even only to detect its presence. In some embodiments, no carried identification device is required in order to capture the information regarding the identity of the healthcare professional using radar technology.

In some embodiments, the reading device is programmed to compare the information captured, in particular information gathered and transmitted by reading the identification device, with information stored in the reading device or in another storage device of the medical treatment system. If, alternatively or additionally, biometric data or signatures or other information is gathered which is not available on an identification device, it may be provided to compare it with stored biometric data or signatures.

In some embodiments, the reading device is programmed to capture, - preferably in a contactless manner - data or information (hereinafter also referred to in short as information) of arbitrary, but different classes, particularly as disclosed herein, such as the gait pattern of the wearer of an identification device, on the one hand, and a graphic representation of a logo of the hospital on a name tag, on the other hand. It can be provided that in order to achieve a result either the information from only one, two or more of these classes or from all of the read-in information must fulfil predetermined requirements. In this way, it can be ensured that the identity of a person, or his or her authorization, can be determined even if, for example, the name tag worn on the gown cannot be recognized because of files carried, a jacket over the gown, etc. but the gait pattern can be, or that a more rapid identification can be made if it is recognized that a person with a stored gait pattern is also wearing at least one hospital logo, for example, on his or her name tag. Moreover, in some embodiments, the certainty with which an identified person has actually been correctly recognized may be increased. If information of several classes is queried, this can be done simultaneously, successively or overlapping.

In some embodiments, the reading device is programmed to obtain information by requesting personal information from the healthcare professional and to compare it with information stored in the reading device or elsewhere in the medical treatment system. Personal knowledge may include, for example, a user name/password combination, other combinations of — for example, logical — sequences and/or answers to personal questions, such as what the mother’s maiden name is or a pet’s name.

In some embodiments, the reading device is programmed to determine the identity of the healthcare professional and/or authorize them to perform certain activities and/or movements based on the result of the comparison.

The information obtained by querying personal knowledge or by reading the identification device can, in some embodiments of the present disclosure, be compared with information available on a server and/or in a database and/or a data storage (device) (hereinafter also referred to as short: server). In these embodiments, the identity of the healthcare professional and/or the authorization to perform certain activities and/or movements is confirmed by positively comparing the query and/or read-out with information available on the server.

A ‘positive comparison’ in the sense of the present disclosure may be the information gathered and/or requested by the reading device corresponding with the information stored, for example, on one or more servers, one or more databases or one or more data storage (device)s.

Whenever information is referred to herein, which is present and/or saved and/or stored “on the network”, this data is preferably saved, stored and/or present on one or more devices suitable for this purpose, for example one or more servers, one or more databases or one or more data storage devices.

The saving and/or storage of information on, for example, one or more servers, one or more databases or one or more data storage devices may be carried out in such a way that different information, for example on groups of healthcare professionals or movement profiles or other information mentioned in the present disclosure, is or are saved and/or stored on separate servers, databases and/or data storage devices. However, different information may also be saved and/or stored on the same, e.g., one or more server(s), database(s) or data storage device(s) or any possible combination.

In some embodiments, the reading device is programmed to transmit the identity and/or a corresponding authorization of the healthcare professional assigned to this identity to the authorization device. In other embodiments, the reading device is provided and/or suitable to be read out, for this purpose, by the authorization device.

Authorizations, for example access-authorizations or the authorizations to perform certain activities which have been granted to a healthcare professional, can be revoked or terminated at any time. This can be done by overwriting or changing previously granted authorizations, which is also referred to herein as a revocation.

Authorizations can be time-based, like their revocation, and/or dependent on the location where the healthcare professional is located or which location they might visit. For example, time-based authorizations can be granted for a specific time window, for example, after identification. It is reasonable here that it is the length of a work shift (usually about 8 to 9 consecutive hours) after the first identification on the specific day, a few minutes after identification at a treatment device, one hour after the last identification, or similar. Depending on the location, certain zones can be designated for revocation, after entering these locations one or more authorizations are revoked, for example, after entering the infection ward, the perhaps unrestricted access to the transplant ward which the healthcare professional had should no longer be possible and therefore the required authorization for this is revoked. These zones can preferably be defined freely, for example using software solutions in the medical treatment system or a control device thereof. In particular, these zones are determined, for example, by the range of the sensor arrangements (camera, radar) within which the healthcare professional themselves or their identification device is detected.

A revocation can also be provided if the healthcare professional comes into contact with someone with whom they should not have and this contact means they should no longer maintain their authorization. This can be particularly important in times of increased risk of infection (flu wave, corona wave, measles wave, epidemic, pandemic, etc.).

For example, it may be provided that a patient who is presumed or proven to be infected with a certain type of virus or bacterium, or who is considered infectious due to other circumstances, carries this information, e.g., with the notification “infectious”, on an identification device assigned to them. This could, for example, be on a marker worn on pyjamas as an example of an identification device of the medical treatment system according to the present disclosure. Such a marker may have any features in connection with the identification device as described herein, in particular those features which enable and/or contribute to the use of the marker (alternatively: marking system) described herein. Through using the devices, systems, and methods described in the present disclosure, safety in the clinical environment can for example be increased, in that, for example, in order to protect other patients and/or nursing staff and/or to prevent further spread of germs in general, the present devices, systems, and methods can be used to determine who is or has been in a room with this patient or near this patient, or can be warned about being near this patient.

If this affects healthcare professionals, e.g., doctors or nursing staff who are still present at other sensitive wards, for example natal units or pediatric wards, intensive care units, transplant wards or the like, the devices, systems, and methods of the present disclosure can determine, for example, that a revocation of authorizations takes place when the reading device or a sensor connected to it detects that both the healthcare professional in question and the patient marked as “infectious” — or another healthcare professional, who was, for example, previously present in the infection’s ward — who are or have been in the same room, in front of the same camera, maintaining less distance with each other than the required minimum distance, etc., especially at the same time. The healthcare professional can then be prevented from entering certain areas of the clinic/practice to which they previously had access, via a notification and/or by withdrawing authorizations.

When this authorization will be again granted, whether a disinfection (single or multiple) carried out in the meantime, a change of clothes or the like is sufficient, or whether (in the worst case) a quarantine and/or a test for the germ is necessary, before work can be resumed or the revoked authorization can again be granted, will have to be decided on a case-by-case basis, or according to predetermined, established rules, for example. The storing of such a “quarantine time” may also be provided for in the medical treatment system according to the present disclosure. The corresponding healthcare professional may then, for example, be completely denied access to the clinic/practice. Such measures advantageously restrict (or ideally prevent) the spread of the germ.

The medical treatment system according to the present disclosure may therefore comprise more than just one identification device. In addition to identification devices which are provided to be worn by medical staff, for example, and which by being worn or carried in interaction with the authorisation device may allow or prohibit one or more activities using or on the medical apparatus and/or one or more movements of the healthcare professional in the vicinity of the medical apparatus, it may also comprise one or more identification devices which are provided to be worn by patients or visitors for example, and which preferably do not give any authorization, but are included by the treatment system according to the present disclosure in its function in order to trace the location or routes of its wearer, now or in the past, e.g., in the vicinity, and to set further identification devices (e.g., markers, marking systems, etc.) in relation to locations or routes of the wearer in order to formulate results therefrom, e.g., to revoke or change authorizations. With the latter the wearer may be notified that their authorization is about to be revoked. The notification may be information or may contain instructions for action. The instruction for action, in so far that it is followed, may optionally result in the authorization not actually being revoked.

In some embodiments, another trigger for revoking authorizations is the registration of an unidentified person by the medical treatment system. For example, if the person cannot be identified by the reading device after being detected by a sensor arrangement. A healthcare professional, for example, who is authorized to access a treatment room and the treatment devices located therein can be denied access to the functions of the blood treatment apparatus for security reasons if an unidentifiable person is detected in the vicinity of the treatment device, e.g., a visitor whom the healthcare professional has previously taken into the treatment room on the basis of his authorization.

In several embodiments the identification device is or comprises a (chip or magnetic) card, an identification tag (badge), a bracelet, an implant, clothing (or is attached to such), a visual aid, e.g., glasses or contact lenses, shoe components or insoles, accessories therefor, graphic prints, for example, as a sticker with a code, active or passive transponders and/or a hand-held device for example a smartphone, a pager, a PDA (Personal Digital Assistant) and so forth, in order to gather the respective information from it.

Graphic prints can, for example, be realized as stickers that represent a code (e.g., barcode or QR code). These stickers can be attached to clothing, for example a gown, in a clearly visible position. Bracelets with such codes are also encompassed by the present disclosure.

Alternatively or additionally to the methods of identification mentioned above, healthcare professionals in the clinic/practice can be identified by a combination of camera and Wi-Fi scanners, WLAN or other devices that are capable of capturing the movement or movement pattern of the healthcare professional. For this purpose, preferably some, several or all of the restricted areas of the clinic or practice and/or the routes between them may be covered by a system of cameras and Wi-Fi scanners, which in turn is connected, for example, to the network of the medical treatment system, clinic or practice. The movement pattern of the passing healthcare professional can be captured and transmitted, e.g., to the server (reading device), where the characteristic signatures of the movement, e.g., the gait pattern, can be compared with stored signatures that can be clearly assigned to the healthcare professional, which can make it possible to identify the healthcare professional in question. The healthcare professional is given the authorizations which correspond to their identity, and subsequently they can act freely within the framework of the authorizations granted to them.

Wi-Fi scanners may, for example, detect identification devices that are logged into an associated wireless network (WLAN) .

In some embodiments, the identification device is integrated into or connected to a pair of glasses which also includes a display section to provide information to the wearer, a camera and means of communication (e.g., Wi-Fi). These glasses can be handed out to healthcare professionals, for example, on entering a clinic/practice. The identification of the healthcare professionals can then be carried out via the camera, e.g., via an eye scan or iris scan, when the glasses are put on and additionally at sensitive locations during the working procedures or before entering them.

Non-contact means for controlling a medical apparatus, for example the treatment apparatus, can be realized using so-called “head-up” displays, for example integrated into the above-mentioned display section of the glasses. A virtual touchpad can be displayed there for example, which can be operated by gesture control or eye movement, which can be, for example, captured by the glasses, e.g., via the camera.

In some embodiments, the medical treatment system also has an issuing and/or collection terminal. This is programmed to create, write and/or read an identification device intended to be carried by the healthcare professional during their time in the clinic or practice.

The issuing and/or collection terminal is preferably programmed to provide the identification device which it creates or writes with information that allows or prohibits its wearer to carry out one or more activities with or on the medical apparatus and/or one or more movements in the vicinity of the medical apparatus, preferably limited with regard to time and/or location.

For example, it may be provided that an identification device is printed out at the issuing and/or collection terminal for a nurse who goes on duty in the morning as a healthcare professional, which he or she attaches to the gown in a clearly visible manner, e.g., as a QR code sticker. The information which can be taken from the QR code by the reading device or the authorization device can authorize the nurse to move to certain places within the clinic (because for example, doors can be opened for (or may be opened by) this healthcare professional on the way due to the identification device they are wearing), for other places, for example due to an infection which the nurse has recently had, they are not (yet) authorized, or not for several days.

It may also be provided, for example, that an identification device (QR code, chip card, etc.) will be issued at the issuing and/or collection terminal for a technician who arrives at the clinic for maintenance on one or more treatment device(s), which entitles him to access the treatment device(s) in question for no longer than that particular working day. Doors and the like on the way there can be opened for him, initiated by the authorization device.

In the event of a later collection or return of the identification devices by the healthcare professional at the issuing and/or collection terminal, e.g., when leaving the clinic/practice, the identification device can be read out automatically if programmed accordingly.

The issuing and/or collection terminal may, thus, be provided to determine how many hours the nursing staff worked that day, or how much time the technician spent at the facility. It is also possible to determine which patients the nurse has attended to and on which treatment facilities the technician has worked, which can also have been recorded in the identification device throughout the day. The latter is particularly advantageous when tracing infection chains or when preparing invoices after the provision of a service.

The issuing and/or collection terminal can be programmed to invalidate or void the identification device after it has been collected/returned or read out.

In some embodiments the treatment apparatus is or comprises an infusion pump, a respirator, an extra corporeal blood treatment apparatus, in particular a dialysis apparatus, a hemodialysis apparatus, hemofiltration apparatus or hemodiafiltration apparatus, in particular an apparatus for acute, chronic renal replacement therapy or for continuous renal replacement therapy (CRRT).

In several embodiments, the diagnostic device is an X-ray apparatus, a computer tomograph (apparatus), a magnetic resonance imaging (MRI) apparatus, an ultrasound apparatus, an ECG apparatus, an apparatus for endoscopy, an apparatus for impedance measuring, an apparatus for thermography, a gamma camera and/or another device for diagnosis, in particular via imaging methods, or comprises at least one of these apparatuses or devices.

In some embodiments, the further clinical medical apparatus is a screen for medical monitoring, a medical refrigerator or a medical cooling compartment, a storage facility for storing disposable medical articles and/or medical instruments, or includes at least one of these machines or apparatuses. The storage facility may, for example, comprise the furniture of the storage unit or parts thereof, i.e., the doors of a cupboard, for example.

Furthermore, the medical clinic apparatuses in several embodiments include, for example, an access door, for example to a ward, an infections department, the clinic/practice, a treatment room, a patient room, a storage room, a social room, a changing room, a locker area, etc.

In some embodiments, the activities are steps or actions for operating, actuating or using the medical apparatus from the group of items, or include such steps or actions, in particular for the intended use of the medical device, e.g., its use during or in the diagnosis or treatment of the patient.

The activities within the meaning of the present disclosure further include, for example, starting or stopping treatment sessions, reacting to alarms, viewing or evaluating diagnoses or examination results obtained via the medical apparatus, and the like. Alternatively or additionally, the activities within the meaning of the present disclosure optionally include those which serve to prepare the medical apparatus for examination, for treatment, etc. or to prepare the patient, for example for a treatment session, such as, for example, causing a cannulation robot to perform a cannulation.

In some embodiments, the movements are or include the healthcare professional approaching the medical apparatus, e.g., the healthcare professional entering into the vicinity of the medical apparatus, or moving away, e.g., leaving the vicinity of the medical apparatus, passing through, for example, a corridor, staircase or the like, entering or leaving a room, for example, or staying in the vicinity of the medical apparatus, by the healthcare professional respectively.

In several embodiments, the vicinity is or includes a predetermined and/or monitored area or a predetermined and/or monitored room of the clinic/practice.

In particular, this is an area which may be entered only with authorization, e.g., only by the medical professional, selected technicians, selected patients or selected visitors, etc.

Alternatively or additionally, the monitoring of an entrance area of the clinic or practice or any other area accessible to the public or also to persons without special access authorization, for example, corridors or staircases, referred to herein as transition areas, is also encompassed by the present disclosure.

In several embodiments, these areas also belong to the vicinity.

Furthermore, an area in which the presence of the healthcare professional at certain times allows the start and/or end of the shift to be defined, for example a changing room, can in several embodiments be monitored and thus be counted as in the vicinity.

Preferably, at least one treatment room, at least one surveillance room, at least one room in which the medical apparatus is placed, at least one room with restricted access, for example a store room, in particular a medicine store room, kitchen, preparation room, etc., and/or the transition area, i.e., the path via a corridor, staircase or the like, from one of the above-mentioned rooms to the other, are monitored and are thus in several embodiments the vicinity or encompassed by the vicinity.

In some embodiments, the medical treatment system according to the present disclosure comprises a writing device or storage device which is programmed to -preferably contactless- write or overwrite data or information on the identification device. When writing or overwriting (data or information) on the identification device information is generated which is readable by the reading device.

In some embodiments, the medical treatment system according to the present disclosure comprises a writing device which is programmed -preferably contactless- to write or overwrite data or information on the authorization device. When writing or overwriting, information is generated which can be read by the reading device.

In some embodiments, if the identified healthcare professional was authorized to perform an initial activity A, they are automatically authorized to perform a further activity B, following activity A. For example, it might be possible, that if the healthcare professional was authorized to enter a treatment room, they are automatically authorized, or given authorization by the medical treatment system to make adjustments, for example to a treatment apparatus. Likewise, other people can be actively excluded from step B and any subsequent steps by not granting or revoking their authorization to perform step B and further steps.

In some embodiments, the conditions for granting authorization may be changed and/or identification requirements minimised in the event of an emergency. For example, in the event of an alarm, any healthcare professional who is and/or was allowed to enter the treatment room may be or may become authorized to make control inputs at the treatment apparatus. Optionally, only inputs that are related to the alarm case can be authorized at the treatment apparatus. The presence of at least one authorized person in the treatment room or in the vicinity of the treatment apparatus can suffice as a prerequisite. See also example (3) as described below.

In some embodiments the identification information can be made anonymous through data pre-processing in such a way that certain requirements, for example, for data protection or for technical data flow or (virtual) storage limitation, can be met. For this purpose, data reduction can already take place during data pre-processing, for example by reducing an image of a face to a facial pattern or an image of a finger to the positions of fingerprint vertices. The remaining data is sufficient to identify the healthcare professional. The data pre-processing can already take place in the electronics of the respective sensors before the data is passed on to the reading device. This means that an identification can still be carried out using the reduced data by, for example, the reading device, however, when reading the identification information by a third party, no conclusions can be drawn about the identified professional.

All medical apparatuses, treatment apparatuses, diagnostic devices and other medical apparatuses may be, within the defined area, in two-way data communication with each other in some embodiments with at least one other of the aforementioned devices. They may also be in two-way data communication via a network with central computers and/or data (base) servers within or outside the defined area, in particular within or outside the hospital. The network can be completely or partially wirelessly connected, for example via WLAN, WiFi, Bluetooth.

Every piece of captured or stored information, whether it be video, audio or other data, can be automatically converted into a format that excludes human use, processing or manipulation. In particular, further monitoring of persons, beyond their identification and authorization, can preferably be prevented.

The following examples are also encompassed within the present disclosure.

A person who is not part of the clinic/practice staff, for example a visitor to a patient, attempts to enter a certain room. As the medical treatment system was unable to identify this person (and probably even less able to determine an appropriate authorization for him or her), the secured door to that room remains closed. A healthcare professional who is nearby may be able to confirm the visitor as such and, for example, after their identification device has been read by the reading device (connected to a camera in front of the door, for example) they are recognized by the authorization device as authorized to enter the room. They can open the door for the visitor or enter the room together with them. Should the visitor consciously or unconsciously wish to tamper with a treatment apparatus in this room, in some embodiments it is not possible for them to do so, as a further authorization is required to operate the treatment apparatus of this example, which also could not be determined for the visitor as the visitor has not been identified as authorized.

A healthcare professional belonging to the clinic/practice staff enters the clinic without being identified or authorized by the medical treatment system according to the present disclosure. However, the staff changing room door requires the entering of a user name/password combination. Even if the healthcare professional had forgotten their password, they could be identified at this point by another identification method, for example by reading aloud a few sentences. Their voice is picked up by a microphone and, therefore, they are identified by the reading device. The healthcare professional can now start their working day as normal, as they receive the authorizations to which they are entitled via the medical treatment system, in particular its authorization device. For example, their shift start-time is recorded automatically, for example in a time recording system, a sticker containing a staff-specific QR code is printed and issued to the healthcare professional. The healthcare professional attaches this sticker to their clothing, for example a gown, so it is visible, and the code is then identified and correspondingly authorized by sensor arrangements with graphic recognition, for example via cameras, on the doors and treatment apparatuses. After the shift, the healthcare professional removes the sticker, for example from the gown, and scans it, e.g., in the changing room, to invalidate it. In some embodiments, this may also involve recording the corresponding end of the shift, for example in a time recording system.

When an emergency situation occurs, e.g., in a treatment room, the identification requirements may be automatically reduced in some embodiments.

For example, if the healthcare professional has left their identification device behind and has no time to search because they are running to the treatment room due to an emergency, their movements are recorded, e.g., by cameras and WiFi sensors on the way there and their identity is identified by the reading device. The authorization device can then allow the door to the treatment room to be opened by the healthcare professional on the basis of this identification alone, and the treatment apparatus where the emergency has occurred can be unlocked ready for input on entering the room. If, however, the healthcare professional in the present stressful situation confuses the control commands of the treatment apparatus and gives the treatment apparatus the command, to shut down for example, the treatment apparatus will not do so because the healthcare professional was not and is not authorized to do so. The healthcare professional is again given the opportunity to enter a — different — command that is suitable for resolving the emergency situation.

In several embodiments, the treatment apparatus is not an implantable medical apparatus.

In some embodiments, the storage device or database is not a part of and/or (not) associated with the treatment apparatus.

In some embodiments, the medical treatment system, the storage device, or the database is not part of the treatment apparatus.

In several embodiments, the medical treatment system is not configured to determine the location or whereabouts of a patient, in particular, it is not configured to determine a relationship between the location of the patient on the one hand and the location of the healthcare professional on the other hand, and most particularly it is not configured to allow or prohibit activities of the healthcare professional based on such a relationship.

In several embodiments, the medical treatment system is not configured to allow the healthcare professional to access information stored in a reading device or in a storage device of the medical treatment system, in particular (not) related to a location of the patient.

In some embodiments, the data or information referred to herein is not, or is not only, data or information from or about the patient, in particular (not) patient data.

In several embodiments, the treatment apparatus and/or the diagnostic device is not a device for emitting radiation or for performing an imaging procedure, in particular it is not a device for performing an X-ray procedure.

In several embodiments, the treatment apparatus and/or the reading device is not adjacent to the door (but is significantly forwards of the door, in order, for example, to make use of a time advantage).

In some embodiments, the identification device is not or does not carry an image or picture of its wearer, or is not linked or associated therewith.

In several embodiments, the medical treatment system or the reading device is not configured and/or programmed so that in order to capture information on the identification device said device has to be moved or swiped through the reading device.

In some embodiments, there is no component on the identification device with coded data or information which is suitable for being read by a closing device or a door and for closing or opening these.

In several embodiments, the identification device does not carry any element or display device which, for example, upon actuation, shows visible changes, should the end of an authorization period be imminent or already reached within a certain period of time, in particular shortly.

In several embodiments, the treatment apparatus and/or its storage device are not configured to capture data or information, in particular kinetic stylometries, of a healthcare professional and archive it for subsequent comparison with other corresponding data or information captured from the same or further or other individuals to enable determining (an) identity or of membership of a particular group.

In several embodiments, the identification device is not configured to detect and/or determine a heart signal, in particular (not) a heartbeat or heartbeat waveform.

Several or all of the embodiments according to the present disclosure may comprise one, several or all of the advantages listed above and/or in the following.

The devices, systems, and methods of the present disclosure can advantageously prevent unauthorized access to medical apparatuses, which may serve to improve the safety of the clinical process (theft, manipulation, contamination, etc.) and indirectly increase the safety of the patient.

The devices, systems, and methods of the present disclosure can advantageously prevent unauthorized access to patients, which can also benefit the safety of the patient, for example because the patient is in an isolation ward and, for example, visitors or even a healthcare professional not dressed accordingly should not have access to the patient.

Through the use of the devices, systems, and methods of the present disclosure, an intentional or unintentional unauthorized operation, (due to lack of authorization) of medical apparatuses of the clinic or practice and/or an initiation of treatment steps (e.g., by placing a handbag or a bouquet of flowers on the buttons of a treatment apparatus) may advantageously be prevented. This can add to the safety of the patient.

Through the use of the devices, systems, and methods of the present disclosure, the effort required to identify clinical personnel when operating identification devices, e.g., on or integrated into medical device(s), can be considerably reduced. This can help to optimise the workflow and save both time and money.

Through the use of the devices, systems, and methods of the present disclosure in connection with necessary hygiene measures, medical safety in the course of the running of hospital operations can be increased, in particular by pre-authorizing the healthcare professional, which may make the use of more than one identification device per day unnecessary.

A further advantage of the present disclosure may be the spatial and temporal separation between the identification of healthcare professional and the operation or use of medical apparatuses. This can help to optimise the workflow in the vicinity and thus save time and money.

Through the use of the devices, systems, and methods of the present disclosure, advantageously common systems for identifying healthcare professionals can be replaced. For example, they can replace keys for opening hospital doors or other traditional means which can easily be lost or misplaced, this can advantageously increase the safety during the work procedures of the healthcare professional but also simplify it. Connecting the identification device - in whatever form - to medical clothing (gloves, gowns, glasses, masks, shoes) also provides protection against loss, as such items regularly remain in the clinic and can be locked away by the healthcare professional, e.g., at the end of a shift.

### BRIEF DESCRIPTION OF THE DRAWINGS

In the following, the present invention is exemplarily explained with regard to the accompanying drawing in which same reference numerals refer to the same or similar components. In the figures of the drawing the following applies:

FIG. 1 shows in a simplified representation, a medical blood treatment system in a first embodiment;

FIG. 2 shows in a simplified representation a blood treatment apparatus and a further clinic apparatus with their respective environment as part of a medical treatment system in a second embodiment;

FIG. 3 shows in a highly simplified representation a treatment apparatus in a treatment room and signifies the monitoring of access to this treatment room via a sensor arrangement;

FIG. 4 shows in a simplified representation a medical blood treatment system in a further embodiment;

FIG. 5 shows a flow diagram of an example for the usage of the medical treatment system described herein in an exemplary embodiment; and

FIG. 6 shows a flow diagram of an example for the usage of the medical treatment system described herein in a further exemplary embodiment.

### DETAILED DESCRIPTION

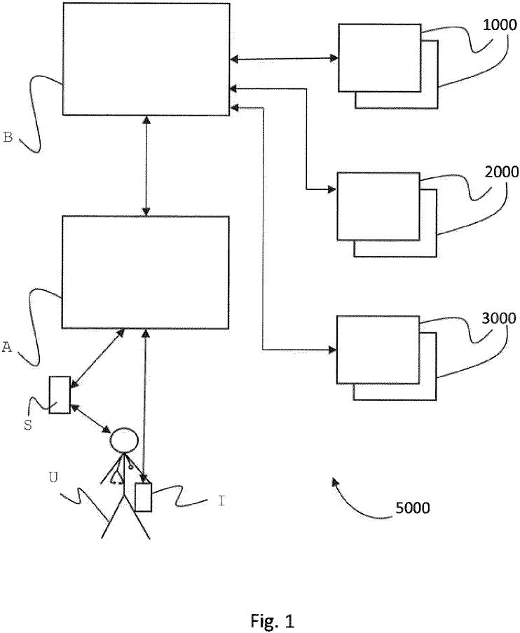

FIG. 1 shows in a simplified representation a medical blood treatment system 5000 in a first embodiment.

It comprises a reading device A, which can be designed as a read-in or read-out device. It is programmed to capture data or information (hereinafter referred to as information) that can serve to identify healthcare professional U and/or to determine their authorization for specific movements or actions. This capturing is preferably contactless.

The healthcare professional U can be a practitioner (doctor) and/or nursing staff (nurse/carer) or belong to the extended hospital staff, for example a (systems) technician, a janitor, a cleaner, etc.

The reading device A is programmed to recognise, determine or establish the identity of the healthcare professional U. For this purpose, it may, for example, access data and/or information originating from or held by an identification device I which the healthcare professional U optionally carries with them.

The identification device I may be, for example, a smart card, a magnetic card, a bracelet, an implant, a badge, and/or a hand-held device. The identification device I may be integrated into the clothing, into a visual aid or the like of the healthcare professional U.

Optionally, the reading device A may additionally or alternatively be programmed to recognize, e.g., via one or more sensor arrangements S, for example biometric information, personal movement patterns, and/or to ask for or gather personal information and to possibly convert it into processable or communicable information. Such information can be captured or generated by the reading device A, and be recognizable to the person skilled in the art, without the healthcare professional U having to carry an identification device I with them, as shown as an option in FIG. 1 as is the case, for example, for gait patterns, physiognomy, etc. Information that has been captured by one or more sensors S can be transmitted by the latter wirelessly, by wired, or in some other way to the reading device A or be read-out via the reading device A. Alternatively, any of the sensor arrangements S mentioned herein can be part of the reading device A or be identical to it.

Furthermore, reading device A, or another device of the treatment system 5000, is also optionally programmed to compare the information it captures — regardless of by which means — with information stored, for example, in it or in a storage medium connected to it, such as a database (not shown in FIG. 1).

The reading device A is programmed to provide and/or to transmit the information which it has captured and possibly processed, to other devices, for example the authorization device B.

On the right in FIG. 1, are shown two (or, independently of one another, fewer than or more than two, e.g., none, one, two or more) treatment apparatuses 1000, diagnostic apparatuses 2000 and clinic apparatuses 3000, hereinafter collectively referred to as a group. The selection of two treatment apparatuses 1000, two diagnostic apparatuses 2000 and two further clinic apparatuses 3000 in FIG. 1 is thus to be understood purely as an example.

The authorization device B is programmed to either allow or prohibit movements and/or activities of the healthcare professional U in connection with or at one of the items of the group (i.e., at least one specific treatment apparatus 1000, diagnostic apparatus 2000 or other clinic apparatus 3000) based on the information captured by the reading device A.

Allowing or prohibiting takes place using the authorization device B, which may be provided together with reading device A, as part of it or independently of it.

On the identification device I, a message or notification related to the authorization may be provided in order to be read by the healthcare professional U. Furthermore, provision may be made to inform the healthcare professional U in this way why, for example, they are not allowed to enter a particular room, e.g., in the form of a text message such as “Lack of protective equipment” or the like.

Permission may be, for example, unlocking an otherwise locked door as an example of another clinic apparatus 3000, so that the professional U who is recognized as authorized can open the door, the door is automatically opened for the professional U, etc. The permission can however be the possibility of the healthcare professional U starting a patient’s treatment using the treatment apparatus 1000, or selecting certain treatment options for which special authorizations are required that should preferably be checked. For diagnostic apparatus 2000 a similar situation may apply.

Prohibiting may, for example, be a item of the above-mentioned group, of the function as a whole or of its ability to be operated by the specific healthcare professional U. In this way for example, an input facility on the treatment apparatus 1000 or the diagnostic apparatus 2000 may be blocked, or when with respect to treatment or diagnosis using this apparatus the (limited) authorization of the professional U is recognized, only a limited range of their generally available options is offered. Similarly, prohibiting can be implemented by blocking access (for example, a locked door) and/or similar measures. A prohibiting can be active or passive. For example, a door (or function) can be actively blocked when approached by healthcare professional U, who is not authorized to pass through it. Alternatively, when the healthcare professional U approaches, the door (or function) can simply remain blocked, i.e., not be unblocked (passive).

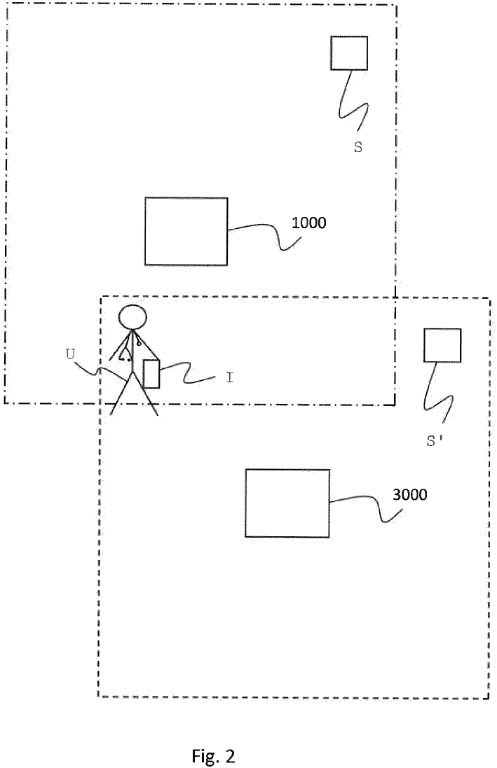

FIG. 2 shows in a simplified representation a treatment apparatus 1000 and a further clinic apparatus 3000 with its respective vicinity as part of a medical treatment system 5000 in a second embodiment.

The randomly defined vicinity of the treatment apparatus 1000 is indicated using a dash-dot line and is detected or monitored by the sensor arrangement S. The vicinity of the other clinic apparatus 3000 is indicated using a dotted line and is detected or monitored via the sensor arrangement S′.

When a healthcare professional U is in the vicinity of a device such as the treatment apparatus 1000 or the other clinic apparatus 3000, the healthcare professional U is identified via the reading device A (see FIG. 1). The information required for this purpose is either captured via the respectively assigned sensor arrangement S, S′, for example a camera, scanner and/or radar, or it is read in by reading information from the identification device I, which the healthcare professional U optionally carries with them.

The information as regards the identity of healthcare professional U is subsequently transmitted to the authorization device B by the reading device A, alternatively, the authorization device B reads the corresponding information from the reading device A (see FIG. 1). For the medical devices in the medical treatment system 5000, here exemplarily the treatment apparatus 1000 and the further clinic apparatus 3000 the authorization device B now determines which authorizations are intended for the healthcare professional U for the respective apparatus 1000, 3000, and issues corresponding authorizations to the medical devices concerned, for example. Such authorizations can, for example, be stored in a storage device of the medical treatment system 5000.

For example, if the healthcare professional U approach the treatment apparatus 1000 for which, in this example, they do not have authorization, authorization device B may result in the healthcare professional U being denied entrance to, or to not having access to treatment apparatus 1000 (alternatively: restricted). This can be done, for example, by simply not opening a door to a room in which the treatment apparatus 1000 is located.

Another example is the blocking of an input at the treatment apparatus 1000, e.g., by locking a screen. In such a case, the limited functionality of the treatment apparatus 1000 is also encompassed by the present disclosure, for example, in that on the screen of the treatment apparatus 1000 only information is displayed that may be read, but no input is possible by the healthcare professional U, e.g., to change treatment parameters. For example, a touchscreen function may be disabled or remain disabled if the healthcare professional U does not have the required authorization.

It may be the case in certain, in particular the present, embodiments that the healthcare professional U is not authorized to use the treatment apparatus 1000, but should very well have access to another clinic apparatus 3000, for example a medicine cabinet in the same room as the treatment apparatus 1000. Authorization device B will therefore allow the healthcare professional U to access the additional clinic apparatus 3000 if the healthcare professional U is identified in the vicinity of and as approaching the additional clinic apparatus 3000. The authorization may be or include unlocking a door interlock to the room where the additional clinic apparatus 3000 is located and/or unlocking the medicine cabinet door.

After their identification by the reading device A, a healthcare professional U, who does not have the required authorization for either of these two apparatuses 1000, 3000, e.g., a facility manager, will not be granted access to either the treatment apparatus 1000 or the other clinic apparatus 3000. Similarly, a healthcare professional U, who has authorization, for example, for both apparatuses 1000, 3000 will be granted access to these by the authorization device B.

This applies equally way to all possible combinations of treatment apparatus 1000, diagnostic apparatuses 2000 and other clinical apparatuses 3000.

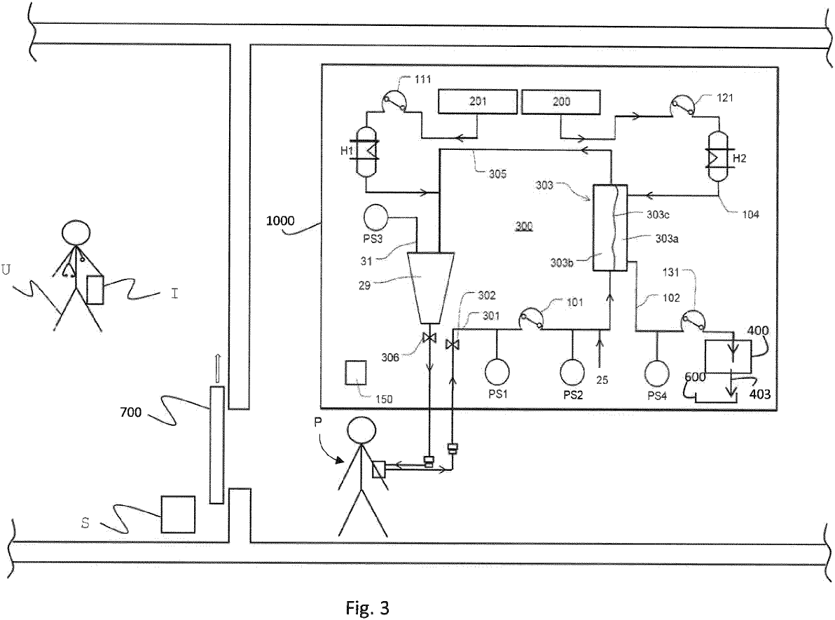

FIG. 3 shows in a highly simplified representation a flow chart of a treatment apparatus 1000, here a blood treatment apparatus, with whose optional, extracorporeal blood circuit 300 a patient P is connected. The treatment apparatus is found in a treatment room (to the right in FIG. 3). The access to this treatment room is cut off via a door 700. The antechamber (to the left in FIG. 3) is monitored by a sensor arrangement S and in this example is already considered to be the vicinity of the treatment apparatus 1000.

FIG. 3 shows the blood treatment apparatus 1000, optionally connected with an outlet tubing system to an optional effluent bag 400

The extracorporeal blood circuit 300 comprises a first line 301, here in the form of an arterial line section.

The first line 301 is in fluid communication with a blood treatment device, here for example a blood filter or a dialyzer 303. The blood filter 303 comprises a dialysis liquid chamber 303a, through which, when in use, dialysis liquid is guided and a blood chamber 303b through which, when in use, blood is guided. The dialysis liquid chamber 303a and the blood chamber 303b are mostly separated from each other by a semi-permeable membrane 303c. Blood and dialysis liquid are mostly guided through the blood filter by the counter current principle. The blood is purified in the blood filter 303.

The extracorporeal blood circuit 300 further comprises at least one second line 305, here in the form of a venous line section.

Both the first line 301 and the second line 305 may be used to connect them to the vascular system of the patient P.

The first line 301 is optionally connected to a (first) tubing clamp 302 for blocking or closing the line 301. The second line 305 is optionally connected to a (second) tubing clamp 306 for blocking or closing the line 305.

The blood treatment apparatus 1000 represented in FIG. 1 only schematically and only by some of its devices comprises a blood pump 101. During the patient’s treatment, the blood pump 101 conveys blood through sections of the extracorporeal blood circuit 300 and towards the blood filter or dialyzer 303, as indicated by the small arrows that generally indicate the direction of flow in each of the figures.

Fresh dialysis liquid is pumped out of a source 200, for example a bag, through an optionally available bag heater H2 with a heating bag, along the dialysis liquid inlet line 104 into the dialysis liquid chamber 303a of a blood filter 303, using a pump for dialysis liquid 121, which may be designed as a roller pump or as an otherwise occluding pump. The dialysis liquid leaves the dialysis liquid chamber 303a via the liquid outlet line 102 towards the effluent bag 400, as dialysate possibly enriched by filtrate. The filtrate may contain water that has been removed from the blood in the blood filter. Dialysate and filtrate are simply referred to as effluent.

The effluent is, in the prior art, directly discarded via an effluent outlet line 102 or, especially during an acute treatment, fed to an effluent bag 400 and is initially stored therein. After completion of the blood treatment, or in bag draining intervals during the blood treatment (intervals in which the effluent bag 400 is emptied), the effluent is discarded from the effluent bag 400, via a sink line, e.g., into a washbasin or into any other type of sink.

The source 200 may be, for example a bag or a container. The source 200 may also be a fluid line, out of which on-line and/or continuously generated or mixed liquid is provided, for example, a hydraulic outlet or hydraulic port of the blood treatment apparatus 1000.

A further source 201 with substitute fluid may be optionally provided. It may correspond to the source 200 or be a separate source.

In addition to the aforementioned blood pump 101 and the pump for dialysis liquid 121, the arrangement in FIG. 1 further comprises, purely optionally, a series of further pumps, in each case optional, namely the pump 111 for substitute fluid, and the pump 131 for the effluent.

The optional pump 111 is provided to feed substitute fluid , from an optional source 201, for example a bag, via an optional available bag heater H1 having a heat bag to the second line 305.

The optional pump 131 is provided to pump effluent out of the blood filter 303 and to convey it in the direction of the effluent bag 400.

Upstream of blood pump 101 an optional arterial sensor PS1 is provided. It measures the pressure in the arterial line during the patient’s treatment.

Downstream of the blood pump 101, but upstream of the blood filter 303 and if provided, upstream of an addition site 25 for Heparin, a further optional pressure sensor PS2 is provided. It measures the pressure upstream of the blood filter 303 (“pre-hemofilter”).

Again, a further pressure sensor may be provided as PS4 downstream of the blood filter 303, however preferably upstream of the pump 131, in the dialysate outlet line 102 to measure the filtrate pressure of the blood filter 303.

Blood, which leaves the blood filter 303, passes through an optional venous blood chamber 29, which can comprise a deaeration device 31 and/or a further pressure sensor PS3. The blood is then returned to the patient (re-infused).

A control device or closed-loop control device 150, here only roughly indicated, can be configured to control or regulate the blood treatment apparatus 100o for the purpose of a blood treatment session. In this example it is part of the blood treatment apparatus 100o. In several embodiments the control device or closed-loop control device 150 is separated from the blood treatment apparatus (1000).

The control device or closed-loop control device 150 may be in cable or cable-less signal communication with any of the herein referred to components of the present medical treatment system.

FIG. 3 also shows a healthcare professional U. This may be a member of the nursing staff or a doctor. In the embodiment shown in FIG. 3, the healthcare professional U is detected by the sensor arrangement S. This can be carried out, for example, by a camera using movement patterns or biometric data, or by reading out the information relating to the identity on the identification device I, which the healthcare professional U optionally carries with them. Alternatively or additionally, a voice sample can be recorded by microphone. The information captured by the sensor arrangement S is transmitted via the reading device A to the authorization device B, which based on the authorization status can decide, for example, whether the door 700 is opened in the direction of the indicated arrow or remains closed to the healthcare professional U or, alternatively or additionally, which (none, some, all) functions of the treatment apparatus 1000 are available for the healthcare professional U.

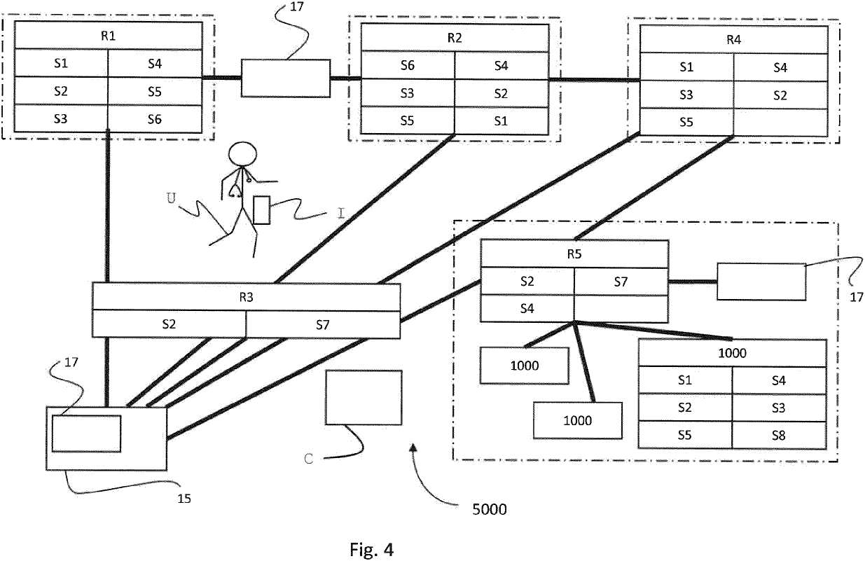

FIG. 4 shows in a simplified representation, a medical treatment system 5000 in a further embodiment.

A healthcare professional U can be identified by various methods. For example, by detecting personal characteristics such as movement patterns, voice, fingerprint, biometric data or their iris structure. In addition, by asking for personal information such as a password or answering a question about the personal details of the healthcare professional U. Another method is to identify the healthcare professional U using an identification device I, for example a magnetic or chip card, a badge or a smartphone, which they optionally carry with them.

Various sensors arrangements S1 to S8 can be used individually or in any combination to capture this information. For example
finger prints are taken using a finger print scanner S1
movement patterns, gestures, biometric data or the iris structure are captured via a camera S2 or via a device S8 for the transmission/receiving of radar waves,
magnetic cards or chip cards are read using a card reader S3,
the voice is picked up by a microphone S4,
personal information is gathered via a touchscreen or touchpad or a keyboard S5,
identification devices I using camera S2, special detection devices S6, WiFi or Bluetooth S7
and transmitted to the reading device A (see FIG. 1)

FIG. 4 shows in the lower left corner a server 15 of the medical treatment system 5000, on which, in this embodiment, both reading device A and authorization device B can be provided. Furthermore, this server 15 can also function as a data server. This is explained below.

As an example, FIG. 4 also shows two, preferably local, databases 17, which can also contain data for determining an authorization. If a person is identified (locally) in these areas, for example, the entrance area R1, changing room R2 and treatment room R5 in FIG. 4, authorizations can be determined quickly and easily using the data stored in the local databases and corresponding actuators can be activated locally.

At least one local database 17 can also exist on server 15 which holds, for example, all the information and only transfers the individual information relevant to the entrance area R1, the changing room R2 and/or the treatment room R5 to the corresponding local databases 17. The local databases 17 can also be mirrored throughout the entire network, so that all databases have identical information. Alternatively, the local databases 17 may also comprise different information necessary for identification in the different areas.

For example, an authorization device B and/or a reading device A, which are provided for gaining access to, e.g., a treatment room R5, can be connected to a local database 17, which only contains information about which specific healthcare professional U is allowed to enter the treatment room R5, which is mentioned by way of example.

A healthcare professional U moves from room to room during their service within the clinic or practice. In this way, they cross so-called transition areas R3, for example corridors, staircases, vestibules, entrances and/or the like.

The entrance area R1 comprises an identification area in which medical workers such as the healthcare professional U should be identified. The healthcare professional U can, for example, be detected via at least one of the existing sensors for the identification devices I, here a fingerprint scanner S1, a camera S2, a card reader S3, a microphone S4, a touch screen or touch pad or a keyboard S5 and/or a special recognition device S6. Thereby, the detecting can be carried out by one or more of the sensors S1 to S6 individually, consecutively or simultaneously. The information captured in this way is, for example, transmitted to a network by the reading device A, for example, in order to store it in one or more servers, in one or more databases or on one or more data storage devices for example, and/or in order to compare it with information that may be stored, e.g., on server 15 or a storage device connected thereto (not shown in FIG. 4) and/or a local database 17. The stored information includes, for example, amongst others, images, voice samples, etc. of the healthcare professional U. Furthermore, the healthcare professional U can be assigned to a group of people, for example, an employee group, e.g., doctors, nursing staff, another professional group, to their general group authorizations and/or individual authorizations of the specific person.

The data server is preferably only accessible from inside the clinic/practice or, additionally or alternatively, from outside.

The network, which can be part of the treatment system according to the present disclosure, comprises for example, as already mentioned above, in addition to a data server an authorization device B, for example a computer, which in connection with suitable actuators can partially control or otherwise influence medical apparatuses such as treatment apparatuses or doors within the clinic/practice.

The information on the data server, for example the access authorization granted to healthcare professional U in connection with their identification, is preferably changeable, i.e., it can, for example, be extended or revoked.

Furthermore, after successful identification of the healthcare professional U, their identification device I, provided they carry one, may be modified in such a way that the authorizations are changed, for example by writing new information into or onto it.

Additionally or alternatively, a current identification device I can be issued to healthcare professional U, containing updated authorizations, e.g., a new badge or a new transponder or similar. The output may be or include printing, printing out, storing, initialising, programming, etc. of an identification device, e.g., a chip card, and may take place, for example, at or via a suitable issuing and/or collection terminal C.

Alternatively or additionally the issuing and/or collection terminal C can be provided as a collection terminal. For example, it may be configured to receive the identification device I and to compare the contents stored on it with the information stored on the network, in order to modify, update and/or the like the contents which are stored on the identification device I and/or on the network, for example stored on one or more servers, on one or more databases or in one or more data memories.

The identification device I may be one of the above or subsequent designs. It is provided, in the course of the working procedures of the healthcare professional U, to enable contactless and rapid identification at relevant points such as, for example, doors, treatment apparatuses 1000, diagnostic apparatuses 2000 or other clinic apparatuses 3000 (see FIG. 1) and/or time capture systems etc.

Alternatively or additionally, the above referred to detecting may then take place, when the healthcare professional U reaches an area that is only accessible to members of the clinic/practice staff, for example a changing room R2. Access is granted here as is described above.

In particular, the issuing of an identification device I may be linked to the issuing of medical clothing. For this purpose, the identification device I may be integrated into the garment. For example, a graphic ID code may be attached to a work coat; another example is work shoes equipped with a radio signal transponder.

Using a camera or radar, scanning the identification devices I which are integrated into the clothing at sensitive locations can, for example, quickly and reliably identify the healthcare professional U there and allow activities via the authorization device B (see FIG. 1), e.g., by unlocking a door, or prohibit activities, e.g. by keeping a door locked.

These places which have limited access, for example the changing room R2, are also covered by the medical treatment system described above using sensor technology in order to be able to identify people who are there as healthcare professional. In some embodiments, there are also issuing and/or collection terminals for Identification devices I, as described above.

Access to an observation room R4 or to a treatment room R5 shall only be granted to healthcare professionals U who have identified themselves at the door to this room, for example by the sensors arrangements S2, S4 and/or S7 having scanned/detected healthcare professional U and the detected information has resulted in an access authorization for this room by an authorization device B. A similar procedure is carried out on the treatment apparatuses 1000.

Advantageously, contactless identification methods are used in observation room R4 and treatment room R5 in order to avoid unnecessary contamination.

The present disclosure makes it possible to determine the authorization of the healthcare professional U, in particular as they are approaching the door, this may minimise unnecessary waiting times.

Three examples are given here of the authorization which the authorization device B issues to the treatment apparatuses 1000:
(a) No further authorization of healthcare professional U is verified at the treatment apparatuses 1000, i.e., authorization to enter the room is regarded as authorization to also operate the treatment apparatus 1000
(b) Contactless determining of authorization by scanning the identification device I of the healthcare professional U. For this purpose, the treatment apparatus 1000 is equipped with a sensor arrangement (e.g., camera S2, card reader S3, radar S8) in order to be able to receive information from the identification device I. The control of the treatment apparatus 1000 then allows all or only some of the functions. The functions of the treatment apparatus 1000 that should be available to the identified healthcare professional U are set by the local database 17. The local database 17 can be accessed locally from the treatment apparatus 1000 or through the network via the corresponding data server 15.In this way, a usage history can be stored in a database, which can record who initiated what activity on the treatment apparatus 1000 at what time.
(c) Identification directly at the treatment apparatus 1000. For this purpose, there is at least one sensor arrangement on the treatment apparatus 1000 (e.g., finger print scanner S1, camera S2, microphone S4, touchscreen or touchpad or keyboard S5). Authorization is then determined in the same way as in example (b). In some embodiments, treatment apparatus 1000 is equipped with a radar in order to recognize (contactless) gestures. These gestures are compared with gestures stored in the database, which in turn can be clearly linked to specific users. The authorization is determined on the basis of the recorded gesture and the result of the comparison.

A wireless connection to certain treatment rooms R5 and the treatment apparatuses 1000 present therein can be established within the observation room R4. Healthcare professional U, for example doctors, can then wirelessly monitor treatment parameters of certain treatment apparatuses 1000 on a display in the observation room R4. In several embodiments, after identification and with appropriate authorization, the healthcare professional U can intervene in the control and/or regulation of the treatment apparatus 1000, i.e., control, from the observation room R4, the treatment apparatus 1000 in the treatment room R5.

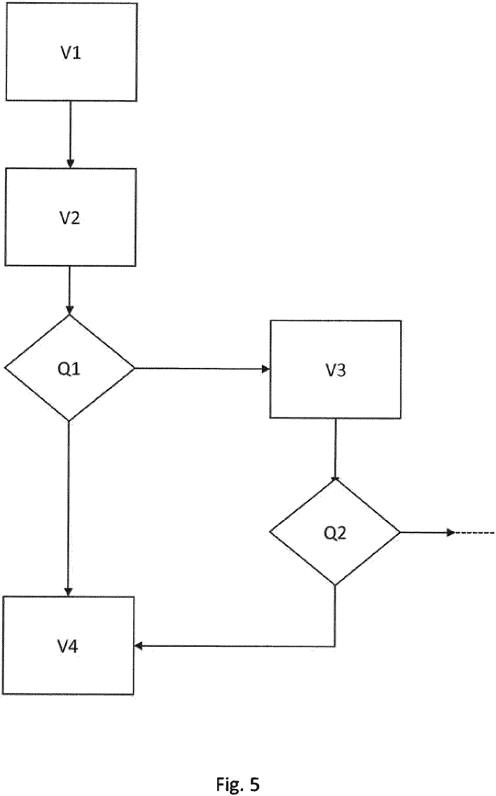

FIG. 5 shows an exemplary flow chart of a usage of the medical treatment system 5000 in an exemplary embodiment.

The healthcare professional U carries an identification device I (see FIG. 1 to FIG. 4) for example an ID badge, a wrist band, or a transponder in or on their shoes. In the clinic/practice entrance area are positioned more sensor arrangements, for example, a card reader S3, a RFID reading device S8, a camera S2 and/or a finger print scanner S1 (not shown in FIG. 5, see FIG. 4).

A step V1 represents the detection or reading out of the identification device I at the entrance to the clinic/practice. If the identification device I is for example a chip card, this can be done via a card reader S3, if it is a transponder for example, this can be done via an RFID reader S8, etc.

In a further step V2 the face of the healthcare professional U should be captured by camera for identification.

The diamond Q1 represents the query as to whether the face was successfully recognized and assigned to the previously identified healthcare professional U by the reading device A.

For example, if the face is covered, reading device A cannot make an identification from the data received from camera S2. This is indicated by the arrow on the right.

For this reason, in a further step V3 the healthcare professional U is requested to initiate a fingerprint scan.

The diamond Q2 represents the query as to whether the fingerprint was successfully scanned and assigned to the previously identified healthcare professional U via reading device A.

If this is not the case, further attempts can be made to identify the previously identified healthcare professional U via further sensor arrangements, which are optionally arranged in this area. Alternatively, it is also possible to fall back on a type of recognition already carried out (here facial recognition, this time with an uncovered face or a fingerprint scan, for example after an intervening cleaning of the scanner). This is represented by the arrow pointing to the right and the subsequent interrupted lines.

If the healthcare professional U has clearly identified themselves in one of the above-mentioned ways (i.e., by an uncovered face or a successful fingerprint scan) (indicated in each case by the arrow extending vertically downwards from the diamonds Q1 or Q2), they may, identified by their identification device I, start the upcoming work period or shift with the necessary authorizations (step V4 in FIG. 5): Using the authorization device B, for example, their working hours are then automatically recorded, doors can be opened automatically when the required authorization of the healthcare professional U is recognized, treatment apparatuses 1000 can be operated by it when the identification device I is an arm’s length or less away from the treatment apparatus 1000, and so forth.

If the maximum working time for that day is reached, the healthcare professional U can be prevented from accessing the treatment room, for example, until the start of the next shift. This can help to reduce or eliminate errors due to fatigue. In addition, the authorization withdrawn in this way can later be used to prove that, for example, a healthcare professional U who may have been inattentive or overtired due to not permissible long working days cannot have worked at the relevant medical apparatuses.

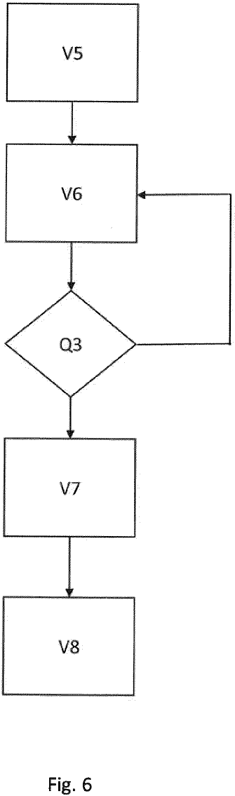

FIG. 6 shows a flow chart of an example of the use of the medical treatment system 5000 in a further exemplary embodiment.

The healthcare professional U would like to change the treatment parameters on a treatment apparatus 1000 in a quarantine station.

In step V5 the healthcare professional U, therefore carries out a complete disinfection and equips themselves with the necessary safety clothing (e.g., gloves, mask, smock, protective goggles) and enters the treatment room R5 (not shown in FIG. 6, see FIG. 4) in the quarantine station. The healthcare professional U cannot identify themselves by fingerprint at the treatment apparatus 1000 because of the gloves they are wearing nor by face recognition because of the face mask they are wearing. Voice recognition is also not possible here due to the damping effect of the face mask. For the reasons mentioned above, gesture movement using radar is the method of choice here.

The execution of a gesture by the healthcare professional U, its detection by radar by reading device A, or its transmission to reading device A is represented in FIG. 6 by step V6.

Via the information transmitted to it, the reading device A can clearly identify or not identify the healthcare professional U by comparing the gesture performed with predetermined gestures already stored. This identification is represented in FIG. 6 by the diamond Q3.

If the healthcare professional U has been clearly identified (shown in FIG. 6 by an arrow pointing vertically downwards from the diamond Q3), the healthcare professional U can be authorized accordingly via an authorization device B and the treatment apparatus 1000 can be released for operation by the healthcare professional U.

In a step V7, the healthcare professional U can now make necessary adjustments or changes to the treatment parameters on the treatment apparatus 1000, for example by speech recognition. The treatment apparatus 1000 may be suitable in just such a case, for clearly recognizing control commands for the treatment despite damping effects of the mask and for causing them to be implemented. A recognition of the voice is no longer necessary at this point, since the healthcare professional U has already identified itself beforehand as described above.

If, in a step V8, the healthcare professional U leaves the treatment room R5, the treatment apparatus 1000 is again blocked to prevent (now unauthorized) entries or changes by the patient, for example.

If the healthcare professional U has not been clearly identified (see diamond Q3 in FIG. 6), the healthcare professional U can, for example, be prompted to perform the gesture again. This is represented in FIG. 6 by a horizontal arrow starting from diamond Q3 and pointing to the right, which leads back to step V6.

List of reference numerals

| | |
|---|---|
| 15 | server; data server; database server |
| 17 | local database |
| 25 | addition point for Heparin (optional) |
| 29 | venous blood chamber (optional) |
| 31 | de-aeration device |
| 101 | blood pump |
| 102 | dialysate outlet line, effluent inlet line |
| 104 | dialysis liquid inlet line |
| 111 | pump for substitute fluid |
| 121 | pump for dialysis liquid |
| 131 | pump for dialysate or effluent in effluent inlet line |
| 150 | control device or closed-loop control device |
| 200 | dialysis liquid source |
| 201 | substitute fluid source, optional |
| 300 | extracorporeal blood circuit |
| 301 | first line (arterial line section) |
| 301′ | (first) line section on the patient |
| 302 | (first) tubing clamp |
| 303 | blood filter or dialyzer |
| 303 a | dialysis liquid chamber |
| 303 b | blood chamber |
| 303 c | semi-permeable membrane |
| 305 | second line (venous line section) |
| 305′ | (second) line section on the patient |
| 306 | (second) tubing clamp |
| 400 | effluent bag |
| 403 | effluent outlet line, sink line |
| 600 | sink |
| 700 | door |
| 1000 | treatment apparatus |
| 2000 | diagnosis apparatus |
| 3000 | further clinic apparatuses |
| 5000 | medical treatment system |
| A | reading device |
| B | authorization device |
| C | issuing terminal and/or collection terminal |
| H1 | bag heater with bag ( substitute fluid ) |
| H2 | bag heater with bag (dialysis liquid) |
| I | identification device |
| P | patient |
| PS1, PS2 | arterial pressure sensor (optional) |
| PS3 | pressure sensor (optional) |
| PS4 | pressure sensor for measuring filtrate pressure |
| R1 | entrance area |
| R2 | changing room |
| R3 | transition area |
| R4 | observation room |
| R5 | treatment room |
| S | sensor arrangement |
| S′ | sensor arrangement |
| S1 | fingerprint scanner |
| S2 | camera |
| S3 | card reader |
| S4 | microphone |
| S5 | touchscreen or touchpad; keyboard |
| S6 | further recognition device |
| S7 | WiFi; Bluetooth |
| S8 | radar; RFID- reader |
| U | healthcare professional |
| V1,..., V8 | process/method steps |
| Q1,..., Q3 | queries regarding with regard to successful capturing and allocation/identification |
| W | writing device |

1-16. (canceled)

17. A medical treatment system, comprising:
a reading device programmed to gather information which serves to identify a healthcare professional, and programmed to provide the gathered information;
a medical apparatus, comprising one or more treatment apparatuses for treating a patient, one or more diagnostic apparatuses for examining the patient, and one or more medical clinic apparatuses; and
an authorization device for allowing or prohibiting one or more activities with or on the medical apparatus and one or more movements of the healthcare professional in the vicinity of the medical apparatus depending on the information provided by the reading device, wherein the one or more movements are those of the healthcare professional entering an area or stepping inside a room.

18. The medical treatment system according to claim 17, wherein the reading device is programmed to capture a movement profile, gait pattern, or gestures of the healthcare professional as information.

19. The medical treatment system according to claim 17, further comprising at least one identification device configured to be worn on the body of the healthcare professional, wherein the reading device is programmed to capture the information by reading the identification device.

20. The medical treatment system according to claim 19, wherein the identification device includes a card, an identification tag, a bracelet, an implant, clothing, a visual aid, shoes, graphic prints, active or passive transponders, and/or a hand-held device.

21. The medical treatment system according to claim 20, wherein the card comprises a chip or magnetic card.

22. The medical treatment system according to claim 19, wherein the reading device is programmed to compare the captured information, which was especially captured and transmitted from the identification device, with information stored in the reading device or in a storage device of the medical treatment system.

23. The medical treatment system according to claim 22, wherein the information stored in the reading device or in a storage device of the medical treatment system comprises biometric data or signatures.

24. The medical treatment system according to claim 17, wherein in order to gather the information the reading device is programmed to request personal information from the healthcare professional and to compare it with information stored in the reading device or in a storage device of the medical treatment system.

25. The medical treatment system according to claim 24, wherein the reading device is programmed to determine the identity of the healthcare professional based on the result of the comparison.

26. The medical treatment system according to claim 17, further comprising an issuing and/or collection terminal programmed to create, write, and/or read an identification device which is to be carried by the healthcare professional, by transferring information to the identification device to allow or prohibit the one or more activities with or on the medical apparatus and/or the one or more movements of the healthcare professional in the vicinity of the medical in a time-limited and/or spatially limited manner.

27. The medical treatment system according to claim 17, wherein the treatment apparatus is or comprises an infusion pump, a ventilator, an extracorporeal blood treatment apparatus, in particular a dialysis apparatus, hemodialysis apparatus, hemofiltration apparatus or hemodiafiltration apparatus.

28. The medical treatment system according to claim 27, wherein the treatment apparatus is an apparatus for acute, chronic renal replacement therapy or for continuous renal replacement therapy (CRRT).

29. The medical treatment system according to claim 17, wherein the diagnostic apparatus is an X-ray device, a computer tomograph, a magnetic resonance imaging (MRI), an ultrasound device, a device for endoscopy, a device for impedance measurement, a device for thermography, a gamma camera and/or another device for diagnosis.

30. The medical treatment system according to claim 29, wherein the diagnostic device is programmed to use imaging methods.

31. The medical treatment system according to claim 17, wherein the medical clinic apparatus is a screen for medical monitoring, an ECG device, a medical refrigerator or a medical cooling compartment, a storage device for storing disposable medical articles and/or medical instruments, or comprises at least one of these devices or apparatuses.

32. The medical treatment system according to claim 17, wherein the activities are or comprise actuating, operating, or using the medical device.

33. The medical treatment system according to claim 32, wherein the activities are for an intended use of the medical apparatus.

34. The medical treatment system according to claim 17, wherein the movements are or encompass, respectively, the healthcare professional approaching the medical apparatus, moving away from the medical apparatus, passing, entering, leaving, and staying in the vicinity of the medical apparatus by the healthcare professional.

35. The medical treatment system according to claim 17, wherein the vicinity of the medical apparatus is or comprises, a predetermined and/or monitored area or a predetermined and/or monitored clinic room, an entrance area, a generally accessible area, an area which may only be entered with authorization, an area in which being present defines the beginning and/or end of work, one or more treatment rooms, one or more monitoring rooms, a room containing the medical apparatus of the group, and/or the route from one to the other of the aforementioned rooms.

36. The medical treatment system according to claim 17, further comprising a writing device programmed to write or overwrite information on the identification device, creating information readable by the reading device.

37. The medical treatment system according to claim 36, wherein the writing device is programmed to write or overwrite information contactlessly.

38. The medical treatment system according to claim 17, further comprising a writing device, programmed for writing or overwriting information on the authorization device, which serves to allow or prohibit the one or more activities with or on the medical apparatus and/or the one or more movements of the healthcare professional in the vicinity of the medical apparatus.

39. The medical treatment system according to claim 38, wherein the writing device is programmed to write or overwrite information contactlessly.
